(19) United States
(12) Patent Application Publication
(10) Pub. No.: US 2007/0106133 A1
(43) Pub. Date: May 10, 2007

(54) Monitoring of vital signs and performance levels

(76) Inventors: Bruce Richard Satchwell, Carrara (AU); Kim Norman Barnett, Mt. Tamborine (AU); Kurt Labes, Elanora (AU); David Meredyth Peeler, Gumdale (AU); Scott Godfrey Cox, Brisbane (AU)

Correspondence Address: ALIVE TECHNOLOGIES PTY LTD, 11 TECHNOLOGY DRIVE, ARUNDEL, 4214, AU

(21) Appl. No.: 10/578,710

(22) PCT Filed: Nov. 18, 2004
(86) PCT No.: PCT/AU04/01620

§ 371 Date: May 10, 2006

(30) Foreign Application Priority Data
Nov. 18, 2003 (AU) 203906345

(51) Int. Cl. A61B 5/00; A61B 5/04

(52) U.S. Cl. 600/309; 128/903; 600/390; 600/323; 600/365; 600/509

(57) ABSTRACT

A monitoring device (10) for monitoring vital signs includes a housing (12). Signal input components (21) are positioned in the housing to receive an electrical signal carrying data representing at least one vital sign of a subject. Wireless communications circuitry (18) is mounted in the housing (12) and is connected to the input components (21) for transmitting and receiving wireless signals.

### FIELD OF THE INVENTION

This invention relates to the monitoring of vital signs and performance levels. More particularly, this invention relates to a device for monitoring vital signs and performance levels and to a system for monitoring vital signs and performance levels.

### BACKGROUND OF THE INVENTION

Monitoring a subject's vital signs is an important aspect of disease control and sports training.

With disease control this is presently generally carried out at a monitoring centre, where a subject can be connected to equipment such as a 12-lead electro-cardiogram (ECG), which is commonly used. For home monitoring, a holter monitor is used over longer periods such as 24 hours to determine if a cardiac event occurs during that time. Other equipment such as oximeters can also be used to monitor blood oxygen level. Pulse oximeters can be used to monitor both pulse rate and blood oxygen level. It will be appreciated that there are many other forms of equipment that can be used to monitor a subject's health and personal activity and performance. Some further examples are blood pressure monitors and glucometers. Still further, by using an accelerometer, it is possible to monitor a patient's orientation and activity level. Using a GPS or other location detection device arranged in some way on a person, it is possible to determine a location of the person.

It has now become possible for subjects to monitor various aspects of their health from home. This is achieved by providing the subject with monitoring devices that are relatively easy to fit and to use. Examples of such devices are ECG monitors, blood pressure monitors and glucometers.

In spite of the fact that there now exists a proliferation of self-monitoring devices, it often remains difficult for a subject to make a decision regarding the result of such self-monitoring. Furthermore, it is difficult for a subject continuously to monitor various characteristics to obtain a pattern that may be important, without being substantially inconvenienced.

Applicant therefore believes that it is desirable that a means be provided whereby a healthcare professional can monitor such characteristics without causing excessive inconvenience to a subject. Still further, Applicant believes that it is desirable that a means be provided whereby a historical record of such characteristics can be collated for analysis at a later date.

As far as physical training is concerned, monitoring of such vital signs as heart rate has long been a standard tool used by coaches to determine the performance and physical capacity of sportsmen and women. In the case of monitoring heart rate, the subject wears a heart rate monitor that can be strapped to the chest. Such monitors are capable of transmitting a signal a metre or two, the signal carrying data representing a heart rate of the subject. It follows that, in order for the coach to be informed of the heart rate, it is necessary for the subject verbally to advise the coach of the heart rate. It will be appreciated that this is not always desirable, particularly in competitive situations, where the subject may not be able to advise the coach of his or her heart rate.

Each subject has a different aerobic threshold. Whether or not a subject has reached his or her threshold can be determined by having knowledge of the subject's optimal heart rate and maximum heart rate. Applicant believes that it would be desirable for a means to be provided whereby a coach could determine the heart rate of a subject without having to communicate directly with the subject. Applicant believes further that it would be desirable for a means to be provided whereby a historical record of a subject's heart rate could be collated for analysis. This applies to other vital signs in addition to heart rate, such as blood-oxygen level, temperature, respiration rate, cardiac output and blood glucose levels.

In the case of training or competing, it is also very useful for a coach to be able to track the movement of an athlete and also the level of activity carried out by the athlete.

Applicant believes that the ability wirelessly to monitor a person's vital signs is highly desirable. There are many reasons for this. For example, wireless monitoring could be used to manage chronic disease to ensure that a chronic condition does not become acute. In this case, wireless monitoring could be used to generate a history relating to one or more vital signs such as heart activity and blood-oxygen level. A medical practitioner could study this history so that a decision can be made as to whether intervention or change of therapy is required.

Monitoring blood composition is essential in the management of blood-related disease. Examples of diseases related to blood composition are diabetes, which is concerned with blood glucose levels and heart disease, which can be concerned with a number of different chemicals such as lipids and cholesterol. There are a number of other blood-related diseases that require monitoring of blood composition.

In the management of diabetes, it is important that levels of blood glucose are monitored on as near to a continuous basis as possible. Various instruments, such as glucometers, are available for this purpose. In order for long term treatment to be effective, patients are required to maintain a record of their blood glucose levels so that the relevant medical personnel can adjust treatment if necessary and calculate long term strategies for management of the disease.

At present, this is done by a patient using the glucometer or similar device to obtain and record a reading. After a predetermined amount of time, the readings are sent to a medical practitioner for graphing and analysis. It will be appreciated that this can be both time-consuming and inefficient. Furthermore, it is not possible for the medical practitioner to provide the patient with real-time evaluation. For example, it would not be possible to advise the patient at the time when a dosage adjustment would be most effective. Applicant has found that such real-time assessment and adjustment is the most effective manner in which to ensure that a patient has long-term benefits from a dosage regime.

At present, if such real-time dosage management were required, it would be necessary for the patient to be in continuous contact with the medical practitioner or be in a treatment centre. It will be appreciated that this is impractical and would place undue hardship on the patient.

Applicant has conceived the present invention in order to obtain a means whereby such real-time management of blood-related diseases can be achieved in an efficient and friendly manner. This would be particularly enhanced with recently available minimally and non-invasive glucometers.

### DEFINITIONS

In this specification, unless otherwise specified, the following words and their derivations will have the associated meanings:

(a) “Subject”—A person whose vital signs are monitored in accordance with this invention.

(b) “Vital sign”—A characteristic relating to the physiology or state of a subject, such as heart rate, blood-oxygen level, respiration rate, state of motion, activity level, position and blood glucose level.

(c) “Wireless communication”—Any communication using a protocol suitable for carrying digital data, such as that known as Bluetooth (trade mark), 802.11a, 802.11b and the more recent “Ultra Wide Band” or UWB, but is not limited to these formats or interfaces.

(d) “Monitoring Centre”—Any location where health monitoring can take place, including not only hospitals, clinics and the like, but also locations where operators receive data relating to vital signs of subjects, depending on the application of the invention.

(e) “Operator”—Any person who monitors health characteristics of the subject. Such a person could be some form of medical practitioner, a sports coach or a fitness instructor or trainer.

(f) “Mobile Phone”—Any communications device that is capable of wireless telephonic communication. Devices such as mobile or cellular phones and personal digital assistants (PDA's) are included in this category.

(g) “Computer”—Any computer-based machine that is capable of executing instructions in a software product. Examples of such machines are a server, a portable computer or a desktop computer. It follows that the definition extends to a number of machines that may define a server.

### SUMMARY OF THE INVENTION

According to a first aspect of the invention, there is provided a monitoring device for monitoring vital signs, the monitoring device including
a housing;
signal input components positioned in the housing to receive an electrical signal carrying data representing at least one vital sign of a subject; and
wireless communications circuitry mounted in the housing and connected to the input components for transmitting and receiving wireless signals.

Processing circuitry may be mounted in the housing, the processing circuitry being configured to process signals generated by the input components and to communicate processed signals to the wireless communications circuitry.

The signal input components may include a number of plug sockets mounted on the housing to permit a number of plugs on electrical leads to be plugged into respective sockets. The processing circuitry may be configured to process signals received from the leads for transmission by the wireless communications circuitry.

The housing may include a first cover member and a second cover member that are configured to be clipped together to enclose the processing circuitry and the communications circuitry. The cover members may be shaped to accommodate the sockets.

The input components may include a number of snap fasteners mounted on the housing and connected to the processing circuitry. The snap fasteners may be spaced to accommodate a number of electrocardiographic electrode studs fastened to a subject.

The housing may include a first cover member and a second cover member that are configured to be clipped together to enclose the processing circuitry and the communications circuitry. In this case, the snap fasteners may be mounted in one of the cover members.

The input components may include a number of metal electrodes mounted on the housing to be accessible from outside the housing and spaced sufficiently to detect an electrocardiographic signal when the electrodes are brought into contact with a subject. Where the housing includes the cover members, the electrodes may be mounted in one of the cover members.

The processing circuitry may include a memory module to permit data representing the signals received by the input components to be stored.

The processing circuitry may be configured to transmit data in the memory module via the communications circuitry.

The processing circuitry may be configured to carry out an analysis on the signals received by the input components to detect anomalies in the signals and to generate a signal for transmission by the communications circuitry on detection of said anomalies.

A discernible signal generating device may be mounted on the housing and may be connected to the processing circuitry. The processing circuitry may be configured to generate a discernible signal for emission by the signal generating device on detection of an anomaly.

A manually operated event switch may be positioned on the housing and connected to the processing circuitry to generate a signal for transmission by the wireless communications circuitry on operation by a user.

A printed circuit board may be mounted in the housing. The processing circuitry and the communications circuitry may be mounted on the printed circuit board.

According to a second aspect of the invention, there is provided a monitoring device kit for monitoring vital signs, the monitoring device kit including
at least two housing members that are detachably connected to each other;
signal input components positioned on one of the housing members to receive an electrical signal carrying data representing at least one vital sign of a subject;
wireless communications circuitry mounted in the housing and connected to the input components for transmitting and receiving wireless signals; and
at least one further housing member that is interchangeable with one of said at least two housing members, further signal input components being positioned on said at least one further housing member.

The at least two housing members may be a first cover member and a second cover member which can be detachably clipped together. The at least one further housing member may be at least one further cover member.

The monitoring device kit may include processing circuitry mounted on the first cover member. The processing circuitry may be configured to process signals generated by the signal input components for transmission by the wireless communications circuitry.

The signal input components may include a number of plug sockets that are connected to the processing circuitry. The first and second cover members may be shaped to accommodate the plug sockets.

The at least one other cover member may be a third cover member. The signal input components may include a number of snap fasteners mounted on the third cover member and connected to the processing circuitry. In this case, where the first and second cover members are shaped to accommodate the plug sockets, the third cover member may be shaped to cover the plug sockets.

Instead, said at least one other cover member may be a fourth cover member. The signal components may include a number of electrocardiographic electrodes mounted on the fourth cover member and connected to the processing circuitry. In this case, where the first and second cover members are shaped to accommodate the plug sockets, the fourth cover member may be shaped to cover the plug sockets.

A printed circuit board may be positioned on the first cover member and a number of spring-mounted contact members may be positioned on the printed circuit board to bear against either the snap fasteners or the electrocardiographic electrodes, depending on whether the third or fourth cover member is attached to the first cover member. The processing circuitry and the communications circuitry may be mounted on the printed circuit board.

According to a third aspect of the invention, there is provided a system for monitoring vital signs, the system including
a monitoring device as described above; and
a receiver for receiving a signal transmitted by the wireless communications circuitry of the monitoring device.

In one embodiment, the receiver may be a wireless modem. The system may include a personal computer that is connected to the wireless modem to receive data relating to the signal. The personal computer may be programmed to carry out algorithmic processes on the data and to display the results of those processes.

The personal computer may be connected to a monitoring centre and may be configured to communicate data relating to the signal received from the monitoring device to the monitoring centre.

In another embodiment, the receiver may be an application-specific device.

In yet another embodiment, the receiver may be a conventional handheld wireless communications device which is configured to receive the signal from the monitoring device and at least to display data relating to the signal to the user. The communications device may be configured to relay the signal to a monitoring centre, via a wireless communications protocol.

According to a fourth aspect of the invention, there is provided a method of monitoring vital signs, the method including the step of receiving data from a monitoring device as described above.

The method may include the step of communicating wirelessly with the subject. In particular, the method may include the step of transmitting a signal to a subject via the wireless communications circuitry of the monitoring device.

The method may include the step of applying analytical algorithms to the data received from the monitoring device.

The method may include the step of downloading data stored in the memory module of the monitoring device via a wireless communications protocol.

According to a fifth aspect of the invention, there is provided an accessory for a monitoring device as described above, the accessory including
a support member;
a number of spaced contact pads positioned on the support member, each contact pad being of a conductive fabric; and
a number of connectors electrically connected to respective contact pads and detachably connectable to the input components of the monitoring device.

The support member may be a sheet of flexible material. The spaced contact pads may be attached to the sheet so that a subject can place both hands on the sheet.

Instead, the support member may be a chest strap, the spaced contact pads being positioned to bear against a subject's thoracic area when worn.

The connectors may be studs to permit the monitoring device to be snap fastened to the support member.

The conductive fabric may be elasticised.

According to a sixth aspect of the invention, there is provided a method of monitoring blood-composition, the method including the steps of:
receiving blood composition data from a sensor;
transmitting a signal carrying the blood composition data to a communications device;
relaying the signal from the communications device to a computer; and
decoding the signal with the computer.

The method may include the step of transmitting data from the computer back to the communications device.

The steps of transmitting signals to and from the communications device may be carried out wirelessly. The step of relaying the signal from the communications device to the computer may also be carried out wirelessly.

According to a seventh aspect of the invention, there is provided a method of treating a patient with a blood-related disease, the method including the steps of:
remotely obtaining blood composition data from the patient at predetermined intervals;
storing the blood composition data in a database;
applying analytical algorithms to the blood composition data when the database is updated; and
sending event-driven signals to the patient based on results of the analytical algorithms.

The step of remotely obtaining blood composition data may include the step of setting up a wireless connection between a blood composition sensor and a communications device and setting up a connection between the communications device and a computer.

According to an eighth aspect of the invention, there is provided an apparatus for monitoring blood composition, the apparatus including
a sensor for sensing blood composition, the sensor being configured to generate a signal carrying data representing the blood composition;
a first communications device connected to the sensor and configured to receive the signal from the sensor and to transmit the signal;
a second communications device that is configured to receive the signal from the first communications device and to transmit the signal; and
a computer that is configured to receive the signal from the second communications device.

The sensor may be a device that is configured to extract a blood sample and to analyse a composition of the blood sample. In a preferred embodiment of the invention, the sensor is a glucometer.

The first communications device may be a wireless interface connected to the sensor. The wireless interface may be configured to communicate according to presently available protocols such as Bluetooth (trade mark); 802.11a; 802.11b and the more recent “Ultra Wide Band” or UWB, but is not limited to these formats or interfaces. The wireless interface and the sensor may have complementary connectors to permit them to be detachably connected together. The wireless interface may have a transceiver to facilitate wireless communication.

The second communications device may be a wireless communications device such as a mobile phone or PDA. The first communications device may be configured to set up a wireless connection with the second communications device. For example, when the protocol used is Bluetooth, the first communications device may be configured to initiate a Bluetooth Serial Port connection to the mobile phone. The mobile phone may be configured to query the glucometer via the first communications device. The mobile phone may also be configured to connect to a computer and to download the results to the computer. In particular, the mobile phone may be configured to connect wirelessly to the computer via a network such as the Internet.

Instead, the first wireless device may be configured to query the glucometer. For example, the first wireless device may be configured to query the glucometer when the first wireless device is powered up. In a particular embodiment, the first wireless device may be configured to be releasably connectable to the glucometer.

In this case, the second communications device may be a wireless access point. Thus, the second wireless device may be a Bluetooth Internet access point or a mobile phone using a Bluetooth Dial Up Network or Link Access Procedure (LAP) protocol. The second device may be configured to make a socket connection to the computer to download the results to the computer.

The computer may be programmed to generate output data for analysis by a medical practitioner. For example, the computer may be programmed to generate graphs or other analytical data to facilitate decision-making by the medical practitioner.

The computer may be in the form of a server on a network such as the Internet. It will thus be appreciated that a patient or a medical practitioner will be able to access the analytical data, via the network, using suitable protocols. It follows that the computer may be programmed to define a web application that can be used by the medical practitioner or the patient.

The computer may be configured to transmit signals back to the mobile phone, via the network. The computer may be configured so that these signal are event-driven or initiated by a medical practitioner. In one embodiment, the computer may be configured so that a medical practitioner can transmit messages to the mobile phone. The messages may use the “Short Message Service” or SMS protocol.

Thus, the system may include an SMS gateway to permit SMS communication between the second communications device and the computer.

According to a ninth aspect of the invention, there is provided an apparatus for monitoring blood composition, the apparatus including
a sensor for sensing blood composition and for generating a signal carrying data representing a blood composition value; and
a communications device that is connectable to the sensor for receiving the signal and for transmitting the signal.

The invention is now described, by way of examples, with reference to the accompanying drawings.

### BRIEF DESCRIPTION OF THE DRAWINGS

In the drawings:

FIG. 1 is an exploded top perspective view of a monitoring device in accordance with the invention.

FIG. 2 is an exploded bottom perspective view of the monitoring device.

FIG. 3 is a top plan view of a circuit board of the monitoring device.

FIG. 4 is an end view of the circuit board.

FIG. 5 is a side view of the circuit board.

FIG. 6 is a bottom plan view of the circuit board.

FIG. 7 is a block diagram of the monitoring device.

FIG. 8 is a schematic diagram of a first embodiment of a system, in accordance with the invention, for monitoring vital signs.

FIG. 9 is a schematic diagram of a second embodiment of a system, in accordance with the invention, for monitoring vital signs.

FIG. 10 is a perspective view of a first embodiment of an accessory, in accordance with the invention, for use with the monitoring device of FIG. 1.

FIG. 11 is a perspective outer view of a second embodiment of an accessory, in accordance with the invention, for use with the monitoring device of FIG. 1.

FIG. 12 is a perspective inner view of part of the accessory of FIG. 11.

FIG. 13 is one embodiment of a system, in accordance with the invention for monitoring blood-glucose level.

FIG. 14 is a device, in accordance with the invention, for monitoring blood-glucose level.

FIG. 15 is a system, in accordance with the invention, for monitoring both a heart rate and a position of a subject.

FIG. 16 is another embodiment of a system, in accordance with the invention, for monitoring blood-glucose level.

FIG. 17 shows a process flow of an embodiment of a method, in accordance with the invention for monitoring blood glucose level.

FIG. 18 shows another process flow of an embodiment of a method, in accordance with the invention for monitoring blood glucose level.

FIG. 19 shows a schematic layout of an apparatus, in accordance with the invention, for monitoring blood glucose level.

FIG. 20 shows a schematic layout of another example of a system, in accordance with the invention, for monitoring blood glucose level.

FIG. 21 shows a schematic layout of a system, in accordance with the invention for monitoring blood-oxygen level.

### DETAILED DESCRIPTION OF THE INVENTION

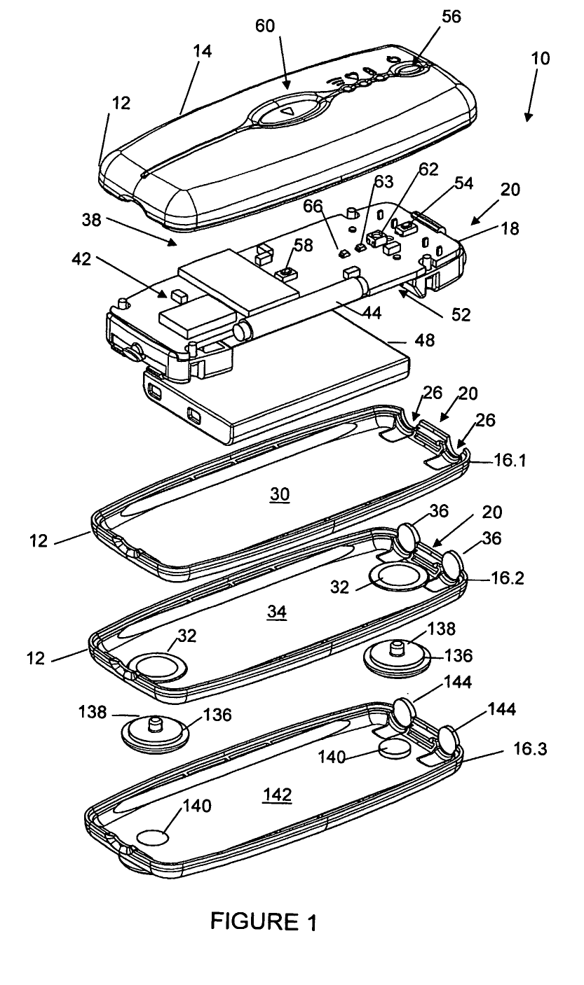

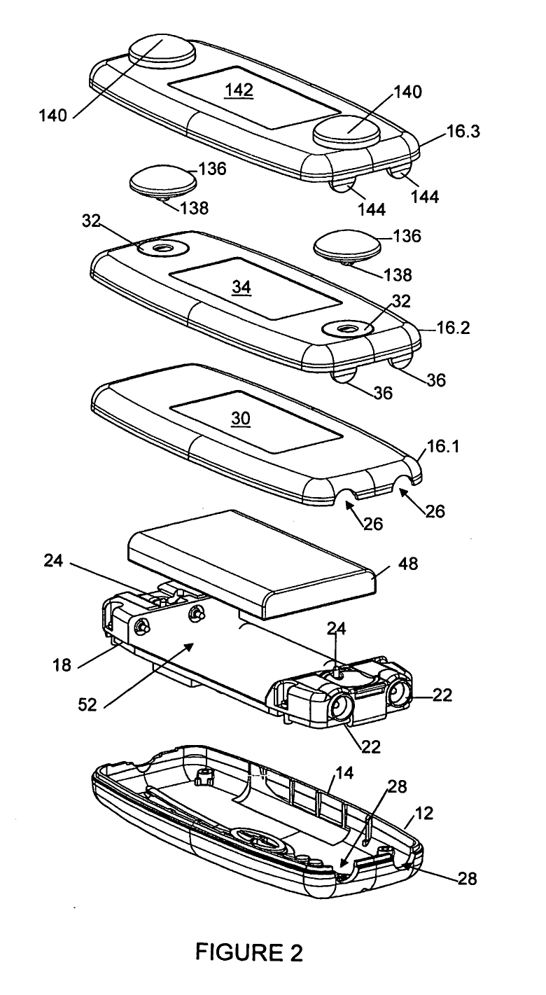

In FIGS. 1 and 2, reference numeral 10 generally indicates a monitoring device or monitor, in accordance with the invention.

The monitor 10 includes a housing 12. The housing 12 includes a top cover 14 and bottom cover 16. A circuit board 18 is interposed between the covers 14, 16 which are fastened to the circuit board 18 with suitable fastening formations 20.

The monitor 10 includes an input means 21 (FIG. 7) in the form of a pair of plug sockets 22 that are mounted on the circuit board 18. The plug sockets 22 are configured to engage plugs (not shown) of leads that are connected to ECG electrodes or any other sensing device for sensing vital signs, such as a glucometer or oximeter. The plug sockets 22 are positioned at an end of the circuit board 18. The input means 21 also includes a pair of spaced, spring-loaded pins 24 that are mounted on the circuit board 18. It follows that the monitor 10 can either receive a signal from the plug sockets 22 or the spring-loaded pins 24.

The monitor 10 is provided with three different forms of bottom cover 16. In a first form, the bottom cover 16.1 has a pair of recesses 26. The recesses 26 correspond with a pair of recesses 28 in the top cover 14 to define a pair of openings for the plug sockets 22. Furthermore, a panel 30 of the bottom cover 16.1 serves to prevent access to the pins 24.

In a second form, the cover 16.2 has a pair of spaced snap fasteners 32 mounted in a panel 34 of the cover 16.2. The snap fasteners 32 are positioned sufficiently far apart to be snap-fastened to respective studs (not shown) of disposable ECG electrodes.

The pins 24 are aligned with the snap fasteners 32 and positioned such that, when the covers 14 and 16.2 are connected together, the pins 24 bear against respective snap fasteners 32. The monitor 10 is configured to be sufficiently light so that when the snap fasteners 32 are connected to the ECG electrodes, the electrodes serve to support the monitor 10 in position without the need for further support.

The cover 16.2 has a pair of tongues 36 that are configured to be received in respective recesses 28 when the housing 12 is assembled. Thus, when the snap fasteners 32 are to be used to receive the ECG signal, the plug sockets 22 are covered. In the alternative configuration, the cover 16.1 serves to protect a wearer against possible electrical shock from exposure to the pins 24.

The device 10 can be supplied with a pair of metal contact electrodes 136. Each contact electrode 136 has a stud 138 that is shaped to clip into one respective snap fastener 32. Thus, it will be appreciated that the device 10 can be used by simply positioning the device 10 against the subject with the electrodes 136 bearing against the subject in a suitable position.

In a third form, the bottom cover 16.3 has metal contact electrodes 140 mounted in a panel 142 of the cover 16.3 to extend outwardly from the panel 142 and also to make contact with the pins 24 when the cover 16.3 is clipped to the top cover 14. In this form, the device 10 can also be positioned against the subject with the electrodes 140 bearing against the subject in a suitable position.

The cover 16.3 also has tongues 144 that serve the same purpose as the tongues 36 of the cover 16.2.

The monitor 10 includes processing circuitry in the form of a microprocessor 38 that is mounted on the circuit board 18. The microprocessor 38 is connected to the input means 21 via an ECG signal amplifier 40 (FIG. 7) to receive an amplified ECG signal from the input means 21.

The monitor 10 includes wireless communications circuitry in the form of a communications module. In this example, the communications module is a Bluetooth (trade mark) module 42. The Bluetooth module 42 is connected to the microprocessor 38 to receive data for transmission from the microprocessor 38 and also to receive signals transmitted to the monitor 10.

The monitor 10 further includes a low-frequency antenna 44 to receive and to transmit signals.

The monitor 10 includes a power supply 46 to power operation of the monitor 10. The power supply 46 includes a rechargeable battery 48 that is connected to the microprocessor 38 and a battery charger 50 that is connected to the battery 48 and to the microprocessor 38 for control of a recharging process.

As can be seen in FIGS. 1 and 2, the battery 48 is engageable with the circuit board 18, via a battery mount 52.

A power switch 54 is mounted on the circuit board 18 and is connected to the microprocessor 38 to permit the monitor 10 to be turned on or off. The power switch 54 is in the form of a push switch that extends through an opening 56 in the top cover 14.

An event switch 58 is also mounted on the circuit board 18 and is connected to the microprocessor 38. The event switch 58 is in the form of a push switch that extends through an opening 60 in the top cover 14. The microprocessor 38 is configured to generate a predetermined signal for transmission by the module 42 when the switch 58 is depressed.

The monitor 10 includes a power status LED 62, a heart status LED 63 and a communication status LED 66 all connected to the microprocessor 38.

In use, the monitor 10 is either fastened to a subject by clipping onto a pair of disposable electrodes that are fastened at a suitable location to the subject or by having electrode plugs received in the plug sockets 22. It will readily be appreciated that the monitor 10 can be fastened to the subject in a number of other conventional ways, if necessary. For example, the monitor could be connected to a strap, as is conventionally used in sport and fitness training. Instead, the monitor 10 could be connected to recently developed “electrode fabric” worn by the subject.

In a medical embodiment, the microprocessor 38 is configured to analyse the ECG or any other signal to detect anomalies, such as atrial fibrillation or a spike in blood glucose levels. Upon the detection of such an anomaly, the microprocessor 38 is configured to generate a signal that is transmitted by the module 42 to a receiver.

In a coaching/training embodiment, the microprocessor 38 is configured simply to transmit the signal to a receiver via the module 42.

The monitor 10 includes a memory module 64 shown in FIG. 7 that is connected to the microprocessor 38. The microprocessor 38 can be configured to store a record of the signal, or characteristics thereof, in the memory module 64. The microprocessor 38 can be configured so that, upon receipt of a suitable signal via the communication or bluetooth module 42, the microprocessor 38 downloads the contents of the memory to a receiver, via the module 42.

It will be appreciated that in a simple form, an operator can communicate directly with the monitor 10 to download data from the memory module 64.

The microprocessor 38 is configured to perform various algorithmic processes on the signal. The results of these algorithmic processes can be stored in the memory module for subsequent download. The microprocessor 38 is re-programmable to alter or re-start the algorithmic processes carried out on the signal. In particular, the micro-processor 38 is configured to receive re-configuration and/or re-programming instructions via the module 42 so that a remote user can re-configure and/or re-program the microprocessor 38.

It will be appreciated that the communications module 42 readily permits communication with the subject. This can be simple communication such as the generation of a sound, via conventional hardware, when the subject is required to take some form of action such as taking a dosage (medical) or slowing down (coaching/training). Instead, the communication can also be vocal, by connecting suitable conventional telephonic hardware to the module 42. This would permit the monitor 10 to be used either for monitoring the signal or for permitting the operator to communicate telephonically with the subject or for both monitoring and communicating.

The monitor 10 can be used as a conventional heart rate monitor. Thus, the microprocessor 38 is configured to receive a signal representing the heart rate from the ECG amplifier 40 via the input means 21 and to generate a signal that carries data representing the heart rate. The monitor includes a conventional short-range radio transmitter 132 that is connected to the microprocessor 38 to transmit the signal to a display 134. The display 134 can, conventionally, be in the form of a wrist display.

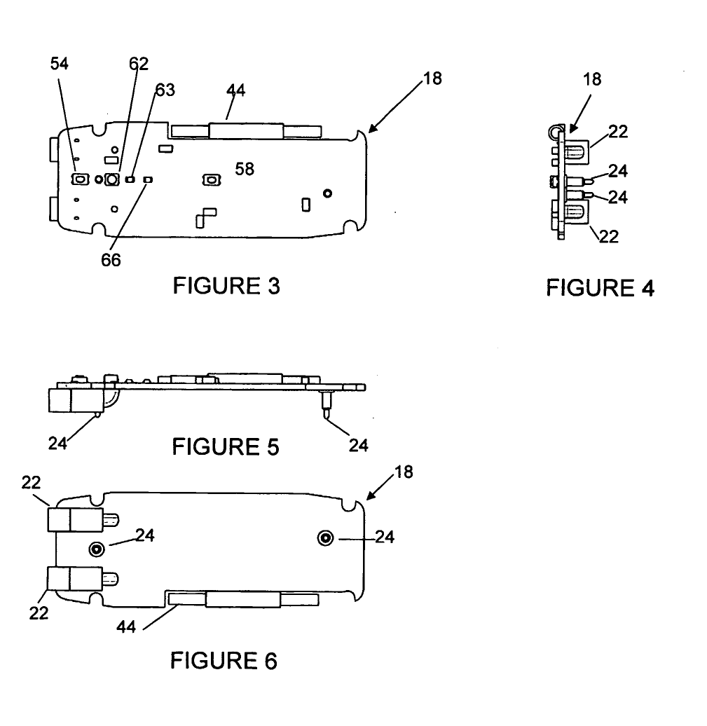

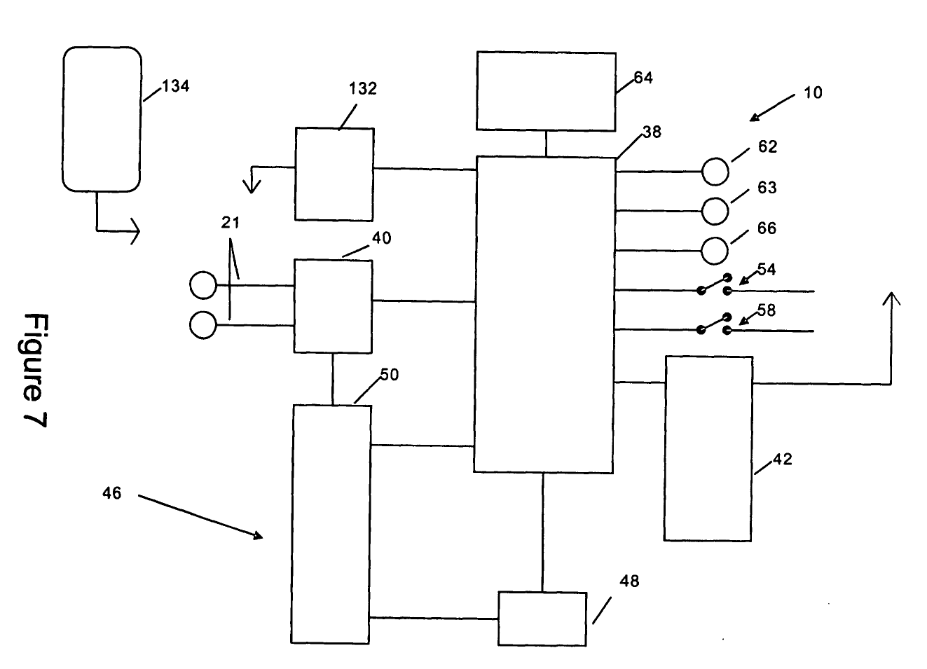

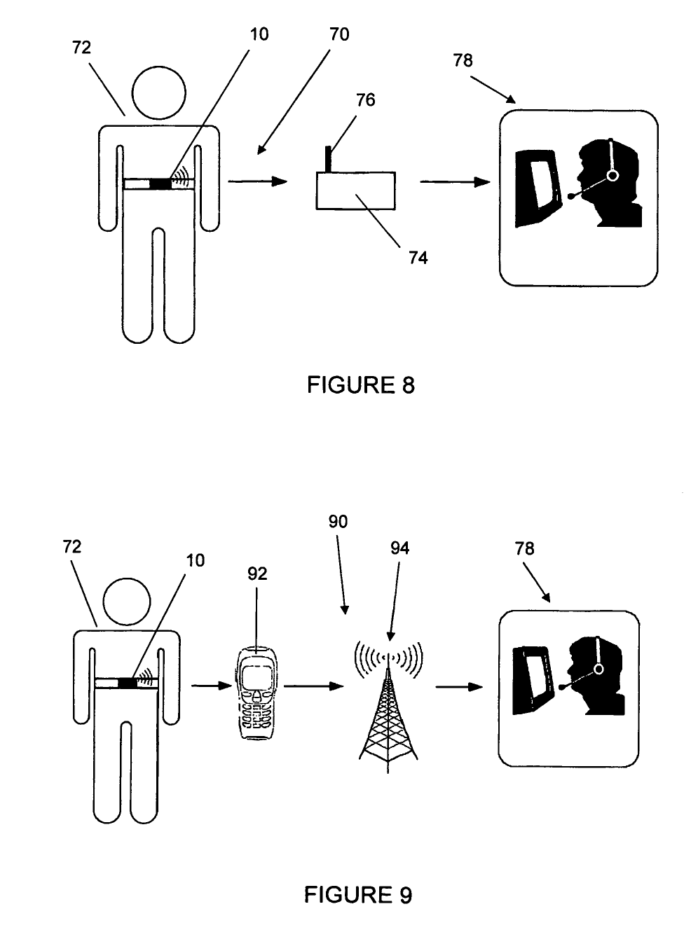

In FIG. 8, reference numeral 70 generally indicates a first embodiment of a system, in accordance with the invention, for monitoring vital signs. With reference to FIGS. 1 to 7, like reference numerals refer to like parts, unless otherwise specified.

In the system 70, the monitor 10 is connected to a subject 72 in any suitable manner, as described above. For example, in the event that the monitor 10 is a heart rate monitor, the monitor 10 can be connected as shown in FIGS. 8 and 9. However, in the event that the monitor 10 incorporates a glucometer, the monitor 10 can be connected to part of the subject 72 for optimal blood glucose testing. This could be in the position shown in FIGS. 8 and 9, for convenience, particularly when the glucometer is non-invasive. The system 70 includes a receiver 74 that is configured to receive and transmit signals to the monitor 10, via the Bluetooth module 42 and a suitable antenna 76. The receiver 74 can be in a number of different forms.

In one embodiment, the receiver 74 is a personal computer (PC). In this case, the PC includes a suitable modem that is connected to the antenna 76 to communicate with the monitor 10. The PC can be programmed to display a visual signal representing the signal. Such a signal is usually only capable of being read by professional operators. Accordingly, the PC can be programmed so that the visual signal is capable of being interpreted by the subject 72. This allows some level of self-monitoring. In certain cases, the PC can be programmed to analyse the signal and to detect anomalies, such as atrial fibrillation or a spike in blood glucose, and to display the presence of such anomalies, also in a form that can be interpreted by the subject 72.

It will be appreciated that the receiver 74 can be provided in a number of different forms depending on the application of the invention. For example, the PC can be portable where necessary, to be used by the operator “in the field” such as when the operator makes house calls on chronically ill subjects or where the operator is a coach or trainer that is monitoring the subject as they train or compete.

In this example, the receiver 74 is connected to a monitoring centre indicated at 78 to communicate with the monitoring centre 78. The manner in which the receiver 74 can be connected to the centre 78 is highly variable. For example, the receiver 74 can be configured to be connected to the centre 78 via the Internet. In another example, the receiver 74 can be wired directly to the centre 78. In yet another example, the receiver 74 can communicate wirelessly with the monitoring centre 78.

In FIG. 9, reference numeral 90 generally indicates a second embodiment of a system for monitoring vital signs. With reference to FIGS. 1 to 8, like reference numerals refer to like parts, unless otherwise specified.

The system 90 is particularly suitable for mobile monitoring of vital signs. In this case, the receiver is in the form of a handheld wireless communications device 92. The device 92 can be provided in a number of different forms. For example, in one form, the device 92 can be an application-specific device that is used by an operator for downloading data from the memory module 64 or simply for recording the signal transmitted by the Bluetooth module 42. In another example, the device 92 is a mobile telecommunications device such as a mobile phone or PDA. The device 92 is configured to receive a signal from the Bluetooth module 42 and to transmit a signal to the module 42.

It will readily be appreciated that the device 92 can be incorporated with the monitor 10, for convenience. Presently available technology provides communication devices which are smaller and lighter than ever before. It follows that it would be relatively simple to incorporate the device 92 with the monitor 10.

In the example shown, the device 92 communicates with the monitoring centre 78 via a mobile relay station network, indicated at 94.

It will readily be appreciated that the systems 70, 90 cover a wide variety of different embodiments that can be used depending on the required application.

For example, the system 70 can be used by an operator at the monitoring centre 78 to communicate with a chronically ill subject to ensure that the subject takes medication on time. The operator can also monitor the subject's vital signs to ensure that the subject can be treated preventatively if necessary. For this purpose, various algorithms can be applied to the data received from the monitor 10 to analyse the data and to instruct appropriate action on such analysis.

The system 90 is particularly useful if used in coaching or training. In such an application, the subject 72 could wear the device 92, together or incorporated with the monitor 10. It follows that the monitoring centre 78 could be a location for the operator in the form of a coach or trainer, who could monitor the vital signs of the subjects and also, as described above, communicate verbally with the subjects.

The system 90 finds useful application in a gym. In this case, the vital signs of a number of subjects could be downloaded to a remote PC or monitoring station. The PC or monitoring station could be networked so that the subject could review his or her performance on the Internet. A trainer could also view the performance of the subject in order to give advice or adjust the training schedule.

Applicant submits that the monitor 10 could be configured to monitor other vital signs, such as blood-oxygen levels. Furthermore, the monitor 10 could be configured to monitor vital signs such as heart rate and blood-oxygen at the same time. With the advent of minimally invasive and non-invasive blood glucose testing, the monitor finds particular application. As a result of this new form of blood glucose testing, the glucometer could be worn by the subject and connected to the monitor 10 via suitable leads in the manner described above. Thus, the blood glucose levels could be remotely conveyed to a trainer or medical practitioner with minimum impact on the subject's lifestyle.

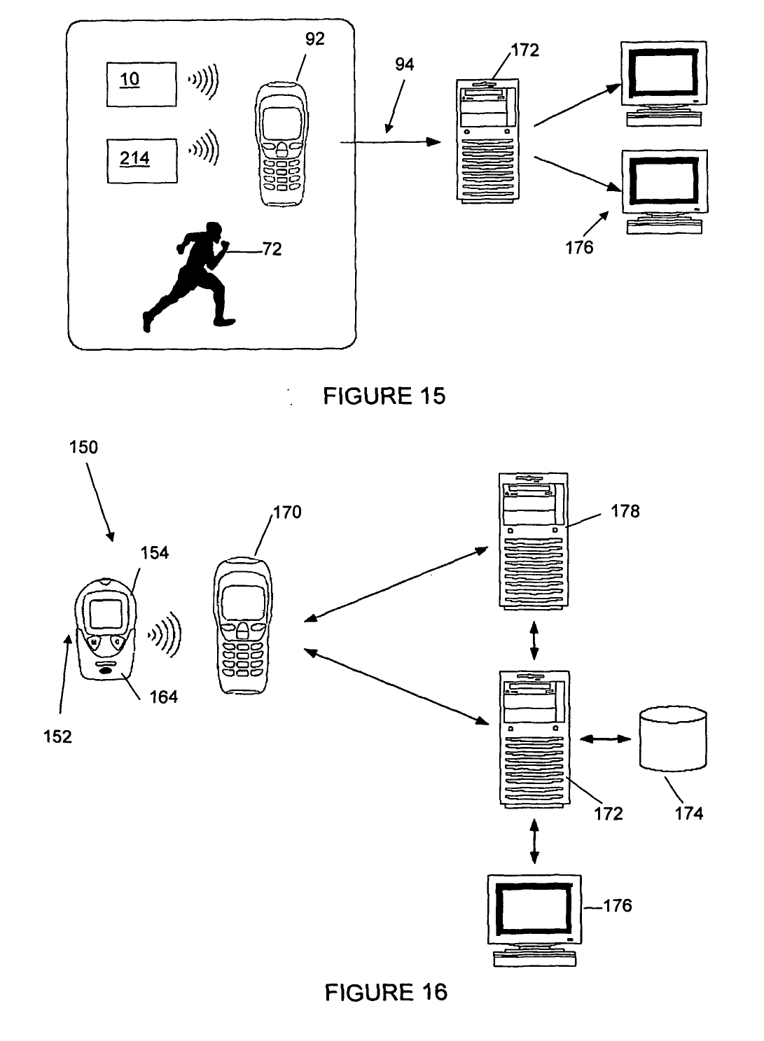

In FIG. 15, there is shown a useful application of the apparatus 10 to the system 90. In this application, the subject 72 uses a GPS receiver 214 in addition to the apparatus 10. The GPS receiver 214 is equipped with a wireless communications module so that a location of the subject 72 can be communicated over the Internet in the manner described earlier.

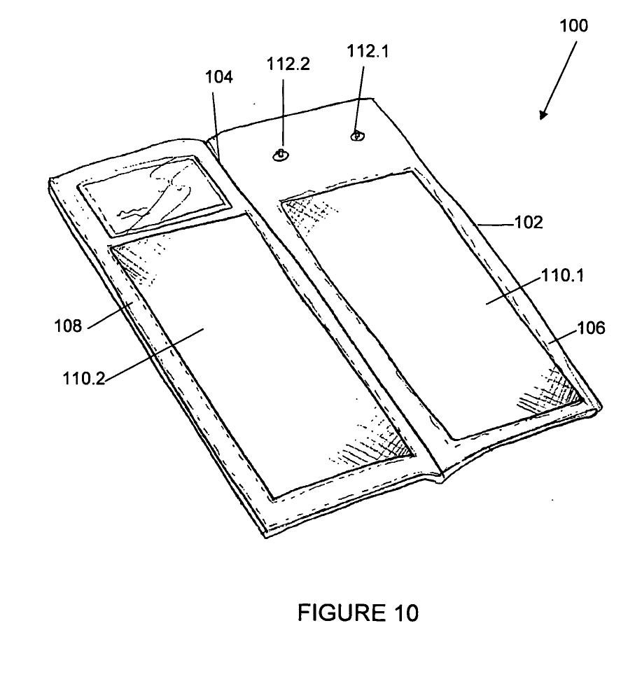

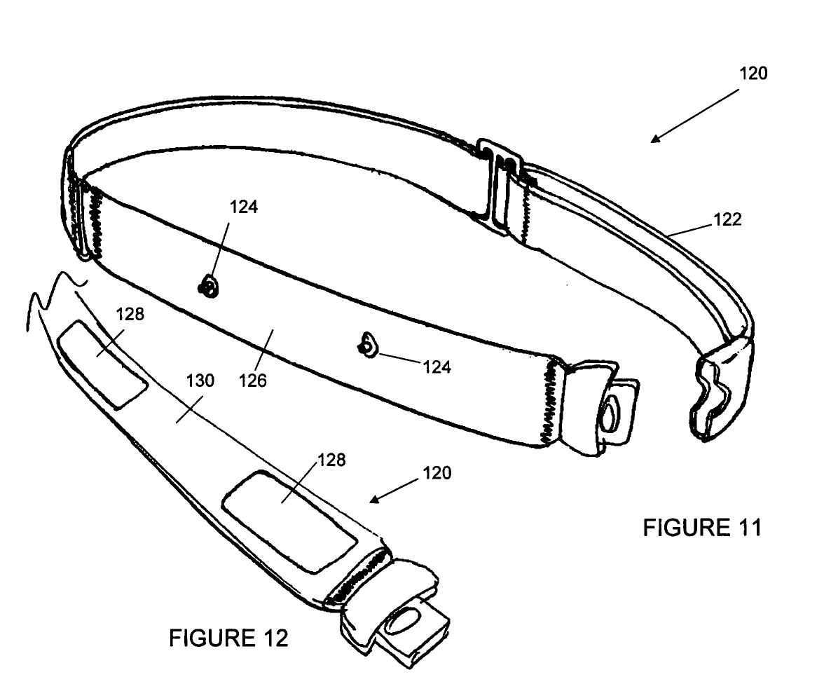

In FIG. 10, reference numeral 100 generally indicates a first embodiment of an accessory, in accordance with the invention, for use with the monitor 10. The accessory 100 includes a support member in the form of a sheet 102 of a flexible material. The material may be leather, vinyl or the like and is configured to have an aesthetically pleasing appearance. Furthermore, the material can be in the form of “smart clothing” which can incorporate the electrodes. It follows that in a particular embodiment, a user can wear clothing that is capable of detecting vital signs, without the need for further components.

The sheet 102 is foldable about a fold line 104 that divides the sheet 102 into a first portion 106 and a second portion 108. The accessory includes a pair of contact pads 110 of a conductive fabric. One of the contact pads 110.1 is positioned on the first portion 106, while the other contact pad 110.2 is positioned on the second portion 108.

A connecting means in the form of a pair of spaced studs 112 is mounted on the first portion 106. Each stud 112 is electrically connected to a respective contact pad 110. The studs 112 are positioned and configured so that the bottom cover 16.2 of the monitor 10 can be clipped onto the studs 112, via the snap fasteners 32.

The contact pads 110 each have elongate tails that extend within the sheet 102 to be crimped to the studs 112. In particular, the contact pad 110.1 is crimped to a left-hand stud 112.2 while the contact pad 110.2 is crimped to a right-hand stud 112.1.

The conductive fabric of the contact pads 110 is a stretch conductive fabric. In particular, the conductive fabric is a medical grade, silver plated fabric. The fabric itself is a combination of Nylon and the fabric known as Dorlastan.

In use, the subject places each hand on a respective contact pad 110. The monitor 10 is thus able to detect the relevant vital signs of the subject via the contact pads 110 and the studs 112.

It will be appreciated that the accessory 100 obviates the need for a subject to wear the monitor 10. In some circumstances, it may not be necessary for the subject to be monitored on a continuous basis. The accessory 100 allows the subject to carry out self-monitoring. Alternatively, an operator can use the accessory 100, where necessary, to obtain data relating to the subject's vital signs.

In FIGS. 11 and 12, reference numeral 120 generally indicates a second embodiment of an accessory, in accordance with the invention, for the device 10. With reference to FIG. 10, like reference numerals refer to like parts, unless otherwise specified.

The accessory 120 includes a support member in the form of a chest strap 122. A connecting means in the form of a pair of spaced studs 124 is mounted on the strap 122 to extend from an outer surface 126 of the strap 122. The studs 124 are shaped and positioned so that the bottom cover 16.2 can be clipped to the strap 122, via the fasteners 32.

A pair of contact pads 128 is positioned on an inner surface 130 of the strap 122. The contact pads 128 are of the same material as the contact pads 110 and are connected to the studs 124 in a similar manner.

A particular advantage of the conductive material used for the conductive pads is that it is absorbent. This allows a certain amount of sweat to be absorbed by the material.

It will be appreciated that the sweat enhances the conductivity of the pads. Furthermore, it is not necessary for the wearer to ensure that the pads are moist, as is the case with presently available heart monitor straps.

Applicant believes that the invention provides a number of significant advantages over presently available equipment. These are based on the fact that the invention provides a means whereby vital signs or data relating to vital signs can be transmitted to a receiver in a form suitable for further transmission or analysis.

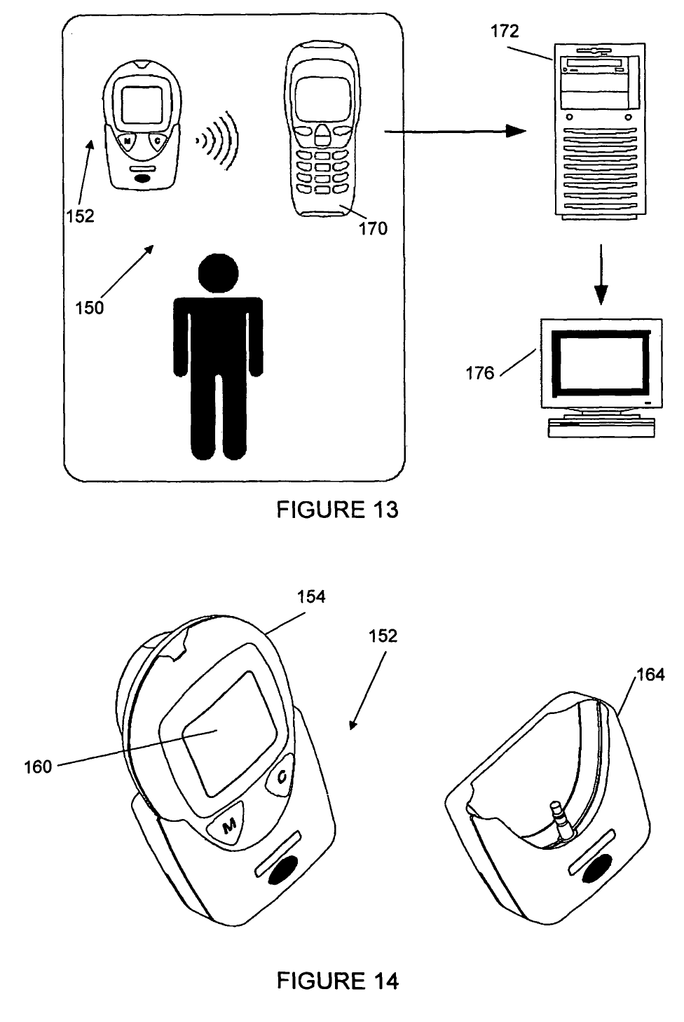

In FIG. 13, reference numeral 150 generally indicates a system, in accordance with the invention, for monitoring blood composition, in particular, blood glucose level.

In this example, the system 150 is configured for monitoring blood glucose. However, it will readily be appreciated that the system 150 can be configured for monitoring other parameters by simply replacing the sensor which is described below.

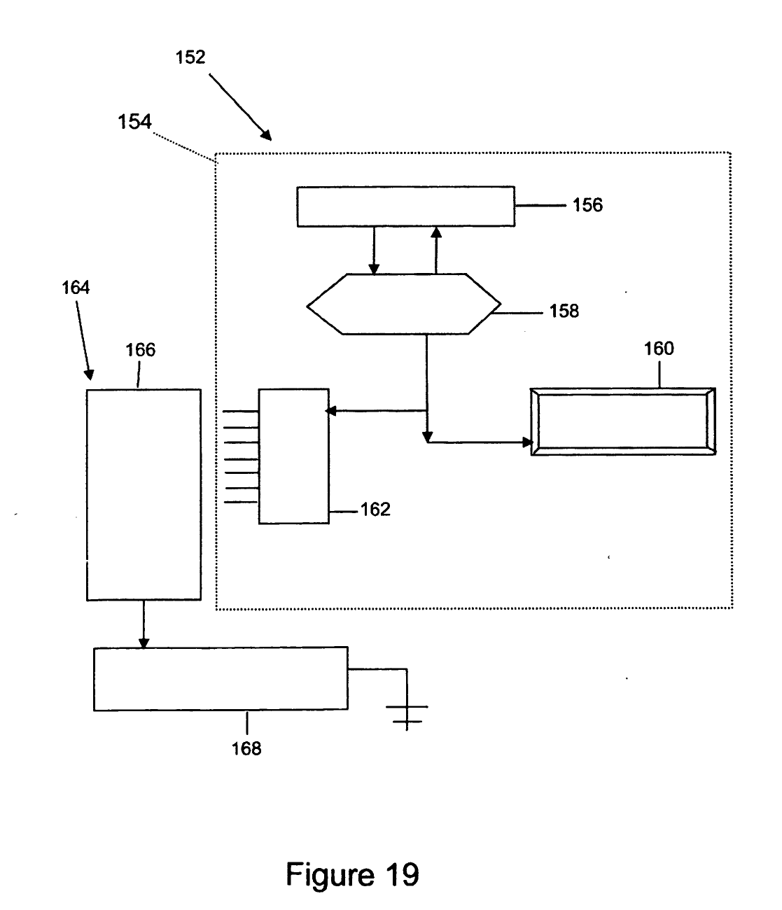

The system 150 includes an apparatus 152, also in accordance with the invention, for monitoring blood glucose level. The apparatus 152 is shown schematically in FIG. 14. The apparatus 152 includes a sensor in the form of a glucometer 154. The glucometer 154 includes a conventional blood glucose reader 156 that detects a level of glucose in a sample of blood. A processor 158 (FIG. 19) receives a signal from the reader 156 and displays a value representing the level of glucose on a display 160 connected to the processor 158.

The glucometer 154 also includes a data connector 162 that receives a signal carrying blood glucose data from the processor 158. The connector 162 can be a socket, pin or other contact arrangement to permit releasable connection of a first communications device described below.

The first communications device is a wireless interface 164 (FIG. 14) for transmitting data to a second communications device described below. The wireless interface 164 is detachably connectable to the glucometer 154. This can be achieved with a complementary connector 166 in the form of a socket, pin or other contact arrangement. The interface 164 includes a transceiver 168 to permit the interface to communicate wirelessly.

The wireless interface 164 is configured to transmit the data using the Bluetooth protocol. It will readily be appreciated that the wireless interface 164 can be configured to use any other protocol, such as those described above.

The system 150 includes a second communications device in the form of a mobile phone 170. In this example, the mobile phone 170 is enabled for Bluetooth communication. The mobile phone 170 is configured to receive the signal transmitted by the interface 164 and to relay the signal to a computer in the form of a server 172.

The server 172 defines a node on a network such as the Internet. Thus, the phone 170 is configured to communicate data received from the interface 164 to the server 172 in a conventional web-enabled manner. The server 172 is configured to store the data in a database 174.

The server 172 can be configured to process the data according to various algorithms. For example the server 172 can be configured to generate historical graphs representing a patient's blood glucose levels. More importantly, the server 172 can communicate with the patient, via the mobile phone 170 using the Bluetooth protocol. Thus, the server 172 can be configured to provide dosage instructions to the patient. These dosage instructions can be provided by the medical practitioner using a web browser indicated at 176. Instead, the server 172 can be programmed to generate dosage instructions depending on the data received from the mobile phone 170. In other words, the server 172 can be configured to provide a dosage that is monitored continuously and that can be adjusted as and when necessary.

As can be seen in FIG. 16, the system 150 can include an SMS gateway 178 to permit SMS communication between the mobile phone 170 and the server 172. Thus, a patient can send results to the server 172 via the SMS gateway 178 and can receive dosage and other communications from the server 172 via the SMS gateway 178.

It follows that the server 172 is programmed to define a web application for performing the operations described above. The web application is also accessible by the patient, via the Internet, so that the patient can obtain analytical information concerning his or her disease.

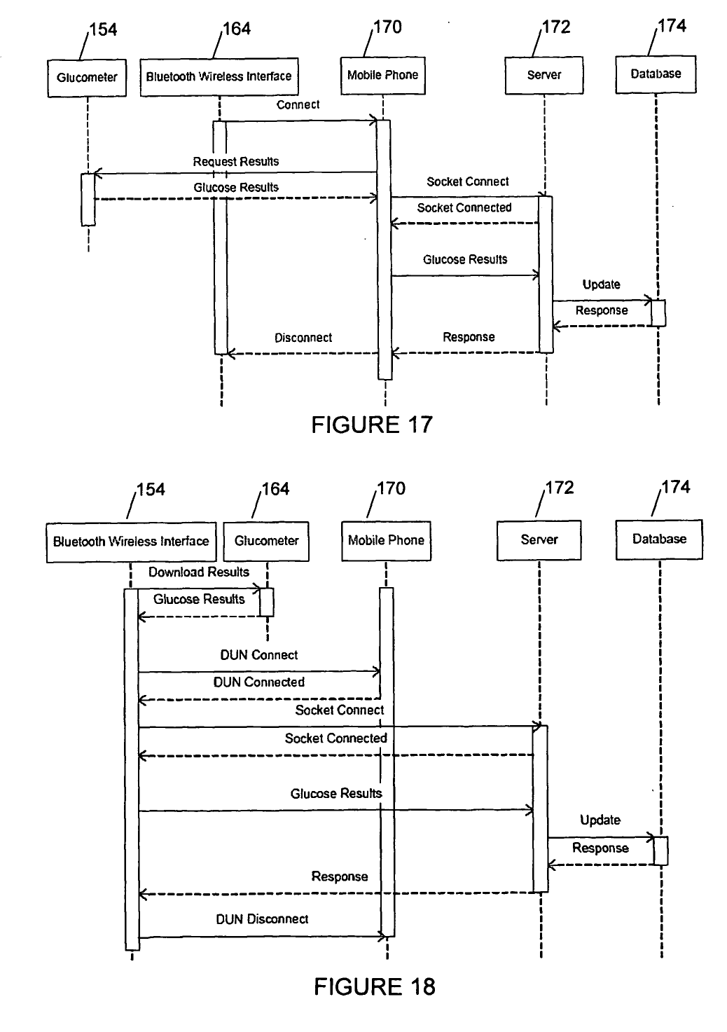

In FIG. 17, reference numeral 180 generally indicates a process flow of a method, in accordance with the invention, for implementing the system 150.

In this method, the wireless interface 164 is connected to the glucometer 154 once a blood glucose measurement has been taken. The interface 164 is configured to initiate a Bluetooth Serial Port (SPP) connection to the mobile phone 170. The mobile phone 170 includes software or firmware that defines an application that queries the glucometer 154 and downloads data representing the blood glucose measurement.

The application sets up a socket connection between the mobile phone 170 and the server 172 via the Internet and downloads the data to the server 172. The server 172 includes software that defines an application that updates the database 174 with the data. The application also sends a response from the server 172 to the mobile phone 170 to indicate that the database has been updated.

In FIG. 18, reference numeral 182 generally indicates a process flow of another method, in accordance with the invention, for implementing the system 150.

In the method of FIG. 18, the patient connects the glucometer 154 to the interface 164 after taking the measurement. The interface 164 includes firmware or software that defines an application that is configured to query the glucometer 154 and to download data representing the blood glucose measurement.

The application then makes a Dial Up Connection between the interface 164 and the mobile phone 170 or between the interface 164 and a second communications device such as a Bluetooth Internet access point. The application then sets up a socket connection between the interface 164 and the server 172, via the Internet, and downloads the data to the server 172. The server 172 includes software that defines an application that updates the database 174 with the data. The application also sends a response from the server 172 to the mobile phone 170 to indicate that the database 174 has been updated.

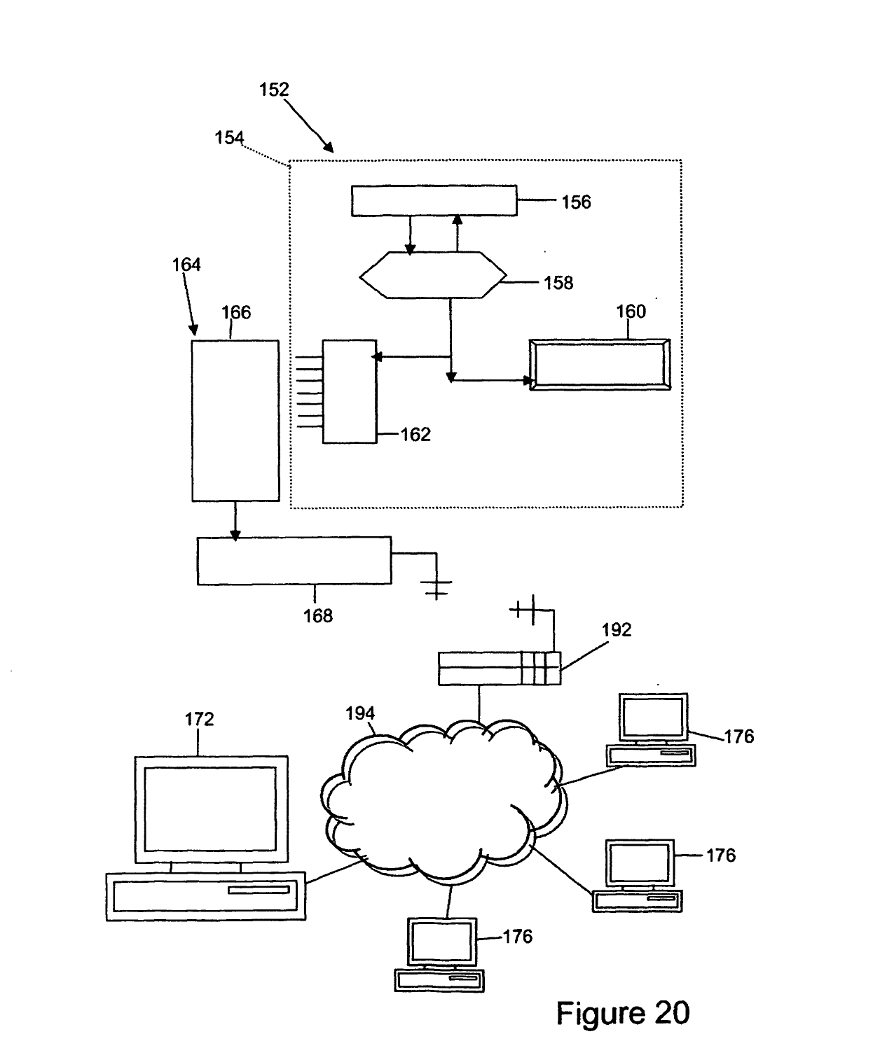

In FIG. 20, reference numeral 190 generally indicates a system, also in accordance with the invention, for monitoring blood composition. With reference to FIGS. 13 to 19, like reference numerals refer to like parts, unless otherwise specified.

The system 190 does not make use of a mobile phone. Instead, the system 190 includes a Bluetooth Internet access device 192 that connects to the Internet 194 using the Bluetooth Dial Up Network or Link Access Procedure (LAP) protocol. Thus, a patient need not have a mobile phone and can have the device 192 positioned at home or any other convenient locations. This would be convenient where the patient has a home-based procedure for taking samples.

In the above example the application can be stored and run on the server 172 and can be configured to provide a number of useful functions.

The medical practitioner can obtain real-time access to blood glucose readings. This allows the practitioner to identify problems at the earliest opportunity. The readings can be presented graphically and numerically, to provide the easiest format for analysis. The application can be configured to run analytical programs so that potential problems can be brought to the practitioner's or patient's attention. The SMS gateway 178 allows the practitioner easily to send messages to the patient, via the browser 176 and server 172.

Applicant believes that this invention provides a means whereby the blood composition of a patient can be monitored on a real-time basis with minimal discomfort to the patient. It follows that with diseases such as diabetes, dosages can be continuously updated so that the patient can achieve long-term benefits which are not readily achievable using the methods available at present. Furthermore, medical practitioners can obtain historical data which is up to date so that quick response is achieved. This response can be managed automatically, in an event-driven manner or can be managed by the medical practitioner.

Still further, the patient can obtain real-time access to the blood composition readings. This allows more involvement in the treatment process by the patient.

An important feature of the invention is the ease with which it can be used. This encourages compliance by the patient with a regime.

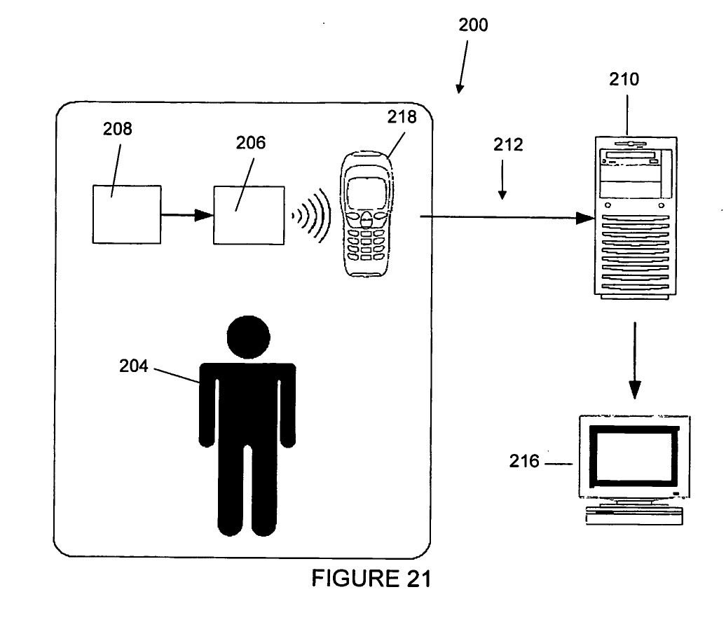

In FIG. 21, reference numeral 200 generally indicates a system, in accordance with the invention, for monitoring a subject's blood-oxygen level.

The system 200 includes a pulse oximeter 202 which can be connected to a subject 204 in a conventional manner. A processor 206 is connected to the oximeter 202 to process the signal from the oximeter 202 into a form suitable for transmission. A communications module in the form of a Bluetooth module 208 is connected to the processor 206 and is configured to transmit the signal wirelessly to a server 210, via a mobile phone 218 and the Internet 212. The server 210 is connected to a web browser 216 so the readings of the pulse oximeter can be monitored. As with the previous embodiments, the server 210 can be programmed to carry out various analytical and event driving processes on the signal received from the oximeter.

Applicant believe that this and the other embodiments of the invention provide a means whereby the vital signs, as defined herein, of a subject can readily and easily be monitored.

1. A monitoring device for monitoring vital signs, the monitoring device including
a housing;
signal input components positioned in the housing to receive an electrical signal carrying data representing at least one vital sign of a subject; and
wireless communications circuitry mounted in the housing and connected to the input components for transmitting and receiving wireless signals.

2. A monitoring device as claimed in claim 1, in which processing circuitry is mounted in the housing, the processing circuitry being configured to process signals generated by the input components and to communicate processed signals to the wireless communications circuitry.

3. A monitoring device as claimed in claim 2, in which the signal input components include a number of plug sockets mounted on the housing to permit a number of plugs on electrical leads to be plugged into respective sockets, the processing circuitry being configured to process signals received from the leads for transmission by the wireless communications circuitry.

4. A monitoring device as claimed in claim 3, in which the housing includes a first cover member and a second cover member that are configured to be clipped together to enclose the processing circuitry and the communications circuitry, the cover members being shaped to accommodate the sockets.

5. A monitoring device as claimed in claim 2, in which the input components include a number of snap fasteners mounted on the housing and connected to the processing circuitry.

6. A monitoring device as claimed in 5, in which the snap fasteners are spaced to accommodate a number of electrocardiographic electrode studs fastened to a subject.

7. A monitoring device as claimed in claim 5, in which the housing includes a first cover member and a second cover member that are configured to be clipped together to enclose the processing circuitry and the communications circuitry, the snap fasteners being mounted in one of the cover members.

8. A monitoring device as claimed in claim 2, in which the input components include a number of metal electrodes mounted on the housing to be accessible from outside the housing and spaced sufficiently to detect an electrocardiographic signal when the electrodes are brought into contact with a subject.

9. A monitoring device as claimed in claim 5, in which the input components include a number of metal electrodes mounted on the housing to be accessible from outside the housing and spaced sufficiently to detect an electrocardiographic signal when the electrodes are brought into contact with a subject, the electrodes being mounted in one of the cover members.

10. A monitoring device as claimed in claim 2, in which the processing circuitry includes a memory module to permit data representing the signals received by the input components to be stored, and the processing circuitry is configured to transmit data in the memory module via the communications circuitry.

11. (canceled)

12. A monitoring device as claimed in claim 2, in which the processing circuitry is configured to carry out an analysis on the signals received by the input components to detect anomalies in the signals and to generate a signal for transmission by the communications circuitry on detection of said anomalies.

13. A monitoring device as claimed in claim 12, in which a discernible signal generating device is mounted on the housing and is connected to the processing circuitry, the processing circuitry being configured to generate a discernible signal for emission by the signal generating device on detection of an anomaly.

14. A monitoring device as claimed in claim 2, in which a manually operated event switch is positioned on the housing and is connected to the processing circuitry to generate a signal for transmission by the wireless communications circuitry on operation by a user.

15. A monitoring device as claimed in claim 2, in which a printed circuit board is mounted in the housing, the processing circuitry and the communications circuitry being mounted on the printed circuit board.

16. A monitoring device kit for monitoring vital signs, the monitoring device kit including
at least two housing members that are detachably connected to each other;
signal input components positioned on one of the housing members to receive an electrical signal carrying data representing at least one vital sign of a subject;
wireless communications circuitry mounted in the housing and connected to the input components for transmitting and receiving wireless signals; and
at least one further housing member that is interchangeable with one of said at least two housing members, further signal input components being positioned on said at least one further housing member.

17. A monitoring device kit as claimed in claim 16, in which the at least two housing members are a first cover member and a second cover member which can be detachably clipped together, the at least one further housing member being at least one further cover member.

18. A monitoring device kit as claimed in claim 17, which includes processing circuitry mounted on the first cover member and configured to process signals generated by the signal input components for transmission by the wireless communications circuitry.

19. A monitoring device kit as claimed in claim 18, in which the signal input components include a number of plug sockets that are connected to the processing circuitry, the first and second cover members being shaped to accommodate the plug sockets.

20. A monitoring device kit as claimed in claim 18, in which said at least one other cover member is a third cover member, the signal input components including a number of snap fasteners mounted on the third cover member and connected to the processing circuitry.

21. A monitoring device kit as claimed in claim 19, in which said at least one other cover member is a third cover member, the signal input components including a number of snap fasteners mounted on the third cover member and connected to the processing circuitry, the third cover member being shaped to cover the plug sockets.

22. A monitoring device kit as claimed in claim 18, in which said at least one other cover member is a fourth cover member, the signal components including a number of electrocardiographic electrodes mounted on the fourth cover member and connected to the processing circuitry.

23. A monitoring device kit as claimed in claim 19, in which said at least one other cover member is a fourth cover member, the signal components including a number of electrocardiographic electrodes mounted on the fourth cover member and connected to the processing circuitry, the fourth cover member being shaped to cover the plug sockets.

24. A monitoring device kit as claimed in claim 18, wherein a printed circuit board is positioned on the first cover member and a number of spring-mounted contact members is positioned on the printed circuit board to bear against signal input components mounted on a further cover member attached to the first cover member, the processing circuitry and the communications circuitry being mounted on the printed circuit board.

25. A system for monitoring vital signs, the system including
a monitoring device as claimed in claim 1; and
a receiver for receiving a signal transmitted by the wireless communication circuitry of the monitoring device.

26. (canceled)

27. A system as claimed in claim 25, in which the receiver is a personal computer that includes a wireless receiver to receive data relating to the signal.

28. A system as claimed in claim 27, in which the personal computer is programmed to carry out algorithmic processes on the data and to display the results of those processes.

29. A system as claimed in claim 27, in which the personal computer is connected to a monitoring centre and is configured to communicate data relating to the signal received from the monitoring device to the monitoring centre.

30. A system as claimed in claim 25, in which the receiver is an application-specific device.

31. A system as claimed in claim 25, in which the receiver is a conventional handheld wireless communications device which is configured to receive the signal from the monitoring device and at least to display data relating to the signal to the user.

32. A system as claimed in claim 31, in which the communications device is configured to relay the signal to a monitoring centre, via a wireless communications protocol.

33. A method of monitoring vital signs, the method including the step of receiving data from a monitoring device as claimed in claim 1.

34. A method as claimed in claim 33, which includes the step of communicating wirelessly with the subject.

35. A method as claimed in claim 34, which includes the step of transmitting a signal to a subject via the wireless communications circuitry of the monitoring device.

36. A method as claimed in claim 33, which includes the step of applying analytical algorithms to the data received from the monitoring device.

37. A method as claimed in claim 33, insofar as claim 33 is dependent on claim 10, which includes the step of downloading data stored in the memory module of the monitoring device via a wireless communications protocol.

38-52. (canceled)

53. A monitoring device as claimed in claim 2, in which an accelerometer is arranged within the housing, and is operatively connected to the signal input components, generating data representing the subject's acceleration.

54. A system as claimed in claim 25, which includes a GPS receiver positioned on the subject, generating GPS signals to the system.

55. A system as claimed in claim 25, in which the receiver is a wireless access device, configured to communicate data relating to the signal received from the monitoring device to the monitoring centre

56. A method as claimed in claim 36, in which the analytical algorithms are applied to measure heart rate variability.
